(19) United States
(12) Patent Application Publication
(10) Pub. No.: US 2005/0192190 A1
(43) Pub. Date: Sep. 1, 2005

(54) Shower oil gels

(76) Inventors: Steffen Hasenzahl, Hanau (DE); Alfred Braunagel, Mainz (DE); Heike Riedemann, Mombris (DE)

Correspondence Address: SMITH, GAMBRELL & RUSSELL, LLP, 1230 PEACHTREE STREET, N.E., SUITE 3100, PROMENADE II, ATLANTA, GA, 30309-3592, US

(21) Appl. No.: 11/068,500

(22) Filed: Feb. 28, 2005

(30) Foreign Application Priority Data
Feb. 28, 2004 (DE) 10 2004 009 812.3

(51) Int. Cl. A61K 7/50

(52) U.S. Cl. 510/130

(57) ABSTRACT

Shower oil gels are disclosed containing up to 70 wt. % of one or more oil-soluble surfactants, 5 to 70 wt. % of one or more oil components, 0.1 to 25 wt. % of one or more pyrogenic silicas and optionally further cosmetic or pharmaceutical auxiliary substances, additives and/or active compounds.

### INTRODUCTION AND BACKGROUND

The present invention relates to a shower oil gel and to cosmetic compositions containing same.

Shower oils comprising 10-90 wt. % of oil, 2-40 wt. % of a gel-forming agent and 0.1 to 20 wt. % of an oil-compatible surfactant are known from BE 08700824.

Shower oils comprising up to 55 wt. % of surfactants together with at least 45 wt. % of one or more oil components from the group consisting of triglycerides of saturated and/or unsaturated branched and/or unbranched fatty acids are known from DE-A-44 24 120. Shower oils are furthermore described in EP 0 867 176 and DE 101 56 674.

Cosmetic or dermatological formulations having a content of hydrophobized inorganic pigments for maintaining the urocanic acid status of the skin are described in DE 44 29 468.

The hydrophobized inorganic pigments are said to prevent the washing out or off, caused by the action of water, of the cis- and trans-urocanic acid endogenous to the skin or applied artificially to the skin.

Shower oils differ from shower gels in that they contain little or no water and, when used, are first rubbed on to the skin, where they then develop, together with water, a cleansing foam which unfolds a skin care and re-oiling action after washing off with the shower water.

The known shower oils have the disadvantage that they are thinly liquid and are therefore difficult to meter. When used, they easily run unused through the fingers. Commercially obtainable shower oils are therefore significantly less viscous than commercially available shower gels, which as a rule are aqueous surfactant formulations, the viscosity of which has been adjusted by addition of salts.

### SUMMARY OF THE INVENTION

The object of the present invention is therefore to develop shower oil gels having a high viscosity. Preferably, these shower oil gels should be transparent and have pseudoplastic or thixotropic flow properties. Furthermore, the foaming power should be controllable.

The invention provides a shower oil gel, characterized in that it comprises pyrogenic silicon dioxide.

The shower oil gels according to the invention can comprise
(a) up to 70 wt %, preferably 5 to 70 wt. % of one or more oil-soluble surfactants
(b) 5 to 70 wt. % of one or more oil components
(c) 0.1 to 25 wt. % of one or more pyrogenic silicas
(d) optionally further cosmetic and/or pharmaceutical auxiliary substances, additives and/or active compounds.

### DETAILED DESCRIPTION OF INVENTION

In a preferred embodiment the shower oil gel can comprise, based on the total formulation, at least 20 to 70 wt. % of one or more oil-soluble surfactants.

The following oil-soluble surfactants can be employed according to the invention:
fatty alcohol sulfates or fatty alcohol ether sulfates, for example TIPA laureth sulfate and MIPA laureth sulfate.
fatty alcohol ethoxylates, for example laureth-3, laureth-4, laureth-2.
fatty acid mono- or diethanolamides, for example coconut fatty acid diethanolamide (cocamide DEA).

The fatty alcohol sulfates or fatty alcohol ether sulfates which are suitable for use according to the invention preferably have the following structure:

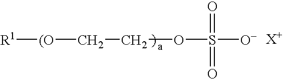

In this formula, “a” can assume values from 0 to 10, preferably 1 to 5, R¹ is selected from the group consisting of branched and unbranched alkyl groups having 6 to 24 carbon atoms;
X⁺ is selected from the group consisting of alkali metal ions and the group consisting of ammonium ions substituted by one or more alkyl and/or by one or more hydroxyalkyl radicals.

The amides of fatty alcohol sulfates or of fatty alcohol ether sulfates which are suitable for use according to the invention preferably have the following structure:

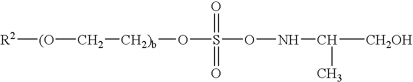

In this formula, “b” can assume values from 0 to 10, preferably 1 to 5; R² is selected from the group consisting of branched and unbranched alkyl groups having 6 to 24 carbon atoms. Preferred fatty alcohol ether sulfates are MIPA laureth sulfate and TIPA laureth sulfate.

The fatty alcohol ethoxylates which are suitable for use according to the invention preferably have the following structure:
R³—(O—CH₂—CH₂—)_{c}—OH

In this formula, “c” can assume values from 1 to 45, preferably 1 to 10; R³ is selected from the group consisting of branched and unbranched alkyl groups having 6 to 24 carbon atoms.

Laureth-4 is the preferred fatty alcohol ethoxylate.

The fatty acid mono- and diethanolamides which are suitable for use according to the invention preferably have the following structures:

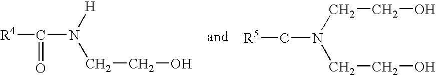

R⁴ and R⁵ in these formulae are selected from the group consisting of branched and unbranched alkyl groups and/or alkenyl groups having 6 to 24 carbon atoms.

Coconut fatty acid diethanolamide (cocamide DEA) is the preferred fatty acid diethanolamide. Naturally occurring coconut fatty acid comprises as essential constituents lauric acid to the extent of 44-51 wt. %, myristic acid to the extent of 13-18 wt. %, palmitic acid to the extent of 8-10 wt. %, caprylic acid to the extent of 6-9 wt. %, capric acid to the extent of 6-10 wt. %, oleic acid to the extent of 5-8 wt. %, stearic acid to the extent of 1-3 wt. %, linoleic acid to the extent of 0-2 wt. % and caproic acid to the extent of 0-1 wt. %.

It is particularly preferable to employ mixtures of MIPA laureth sulfate, laureth-4 and coconut fatty acid diethanolamide. Such mixtures are obtainable, for example, under the name ZETESOL® 100 from Zschimmer & Schwarz Chemische Fabriken, Lahnstein/Rhein, or TEXAPONI® WW 99 from Henkel KGaA, Düsseldorf, Germany.

The wash-active substances in the context of the present invention are preferably selected from the group consisting of wash-active anionic, cationic, amphoteric and/or nonionic surfactants.

Wash-active anionic surfactants in the context of the present invention are acylamino acids and salts thereof, such as acylglutamates, in particular sodium acylglutamate, sarcosinates, for example myristoyl sarcosine, TEA lauroyl sarcosinate, sodium lauroyl sarcosinate and sodium cocoyl sarcosinate, sulfonic acids and salts thereof, such as acyl isethionates, for example sodium/ammonium cocoyl isethionate, sulfosuccinates, for example dioctyl sodium sulfosuccinate, disodium laureth sulfosuccinate, disodium lauryl sulfosuccinate and disodium undecylenamido-MEA sulfosuccinate, and sulfuric acid esters, such as alkyl ether sulfates, for example sodium, ammonium, magnesium, MIPA, and TIPA laureth sulfate, sodium myreth sulfate and sodium C12-13-pareth sulfate, and alkyl sulfates, for example sodium, ammonium and TEA lauryl sulfate.

Wash-active cationic surfactants in the context of the present invention are quaternary surfactants. Quaternary surfactants contain at least one N atom which is covalently bonded to 4 alkyl or aryl groups. Benzalkonium chloride, alkylbetaine, alkylamidopropylbetaine and alkyl-amidopropylhydroxysultaine are advantageous.

Wash-active amphoteric surfactants in the context of the present invention are acyl-/dialkylethylenediamines, for example sodium acylamphoacetate, disodium acylamphodipropionate, disodium alkylamphodiacetate, sodium acylamphohydroxypropylsulfonate, disodium acylamphodiacetate and sodium acylamphopropionate.

Wash-active nonionic surfactants in the context of the present invention are alkanolamides, such as cocamide MEA/DEA/MIPA, esters which are formed by esterification of carboxylic acids with ethylene oxide, glycerol, sorbitan or other alcohols, ethers, for example ethoxylated alcohols, ethoxylated lanolin, ethoxylated polysiloxanes and propoxylated POE ethers, and alkyl polyglycosides, such as lauryl glucoside, decyl glycoside and coco glycoside.

Further anionic surfactants are taurates, for example sodium lauroyl taurate and sodium methylcocoyl taurate, ether-carboxylic acids, for example sodium laureth-13 carboxylate and sodium PEG-6 cocamide carboxylate, phosphoric acid esters and salts, such as, for example, DEA oleth-10 phosphate and dilaureth-4 phosphate, alkylsulphonates, for example sodium coco-monoglyceride sulfate, sodium C12-C14-olefinsulfonate, sodium lauryl sulfoacetate and magnesium PEG-3 cocamide sulfate, and carboxylated, ethoxylated vegetable oils, in particular olive oil PEG-7 carboxylate.

Further amphoteric surfactants are N-alkylamino acids, for example aminopropylalkylglutamide, alkylaminopropionic acid, sodium alkylimidodipropionate and lauroamphocarboxyglycinate, and sodium carboxymethyl cocopolypropylamine.

Further suitable anionic surfactants in the context of the present invention are furthermore acylglutamates, such as di-TEA palmitoyl aspartate and sodium caprylic/capric glutamate, acylpeptides, for example palmitoyl hydrolysed milk protein, sodium cocyl hydrolysed soya protein and sodium/potassium cocoyl hydrolysed collagen, and carboxylic acids and derivatives, such as, for example, lauric acid, aluminium stearate, magnesium alkanolate and zinc undecylenate, ester-carboxylic acids, for example calcium stearoyl lactylate, laureth-6 citrate and sodium PEG-4 lauramide carboxylate, and alkylarylsulfonates.

Further suitable cationic surfactants in the context of the present invention are furthermore alkylamines, alkylimidazoles and ethoxylated amines.

Further suitable nonionic surfactants in the context of the present invention are furthermore amine oxides, such as cocoamidopropylamine oxide.

The oils according to the invention can preferably be selected from the group consisting of polar oils, for example the group consisting of triglycerides or lecithins. The use of polar oils of plant origin, for example avocado oil, cottonseed oil, groundnut oil, thistle oil, jojoba oil, pumpkin oil, kukui nut oil, macadamia nut oil, maize germ oil, almond oil, evening primrose oil, olive oil, palm oil, rapeseed oil, sesame oil, soy bean oil, sunflower oil, wheat germ oil, castor oil, safflower oil or grapeseed oil, may be particularly advantageous.

Paraffin oil or linear and/or cyclic silicone oils can furthermore be used. Suitable synthetic oils can be, for example:
mono-, di- and triesters of linear or branched and/or saturated or unsaturated alcohols and fatty acids having in each case 6-40 C atoms;
ethers between linear or branched and/or saturated or unsaturated alcohols having in each case 6-40 C atoms;
linear and cyclic hydrocarbons having 6-40 C atoms.

Octyl stearate, hexyl laurate, dibutyl adipate, cetearyl isononanoate, decyl oleate, oleyl erucate, caprylic/capric triglyceride, dicapryl ether and/or dioctylcyclohexane are possible in particular.

Further oils which can be used according to the invention are known from DE 101 56 674, page 6, line 9 to 59.

An optionally desired oil component of the cosmetic or dermatological cleansing formulations in the context of the present invention is preferably selected from the group consisting of esters of saturated and/or unsaturated, branched and/or unbranched alkanecarboxylic acids of a chain length of 3 to 30 C atoms and saturated and/or unsaturated, branched and/or unbranched alcohols of a chain length of 3 to 30 C atoms, and from the group consisting of esters of aromatic carboxylic acids and saturated and/or unsaturated, branched and/or unbranched alcohols of a chain length of 3 to 30 C atoms. Such ester oils can then preferably be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and naturally occurring mixtures of such esters, for example jojoba oil.

The oil component can furthermore preferably be selected from the group consisting of branched and unbranched hydrocarbons and hydrocarbon waxes, silicon oils and dialkyl ethers, and the group consisting of saturated or unsaturated, branched or unbranched alcohols and fatty acid triglycerides, namely triglycerol esters of saturated and/or unsaturated, branched and/or unbranched alkanecarboxylic acids of a chain length of 8 to 24, in particular 12 to 18 C atoms. The fatty acid triglycerides can preferably be selected, for example, from the group consisting of synthetic, semi-synthetic and naturally occurring oils, for example olive oil, sunflower oil, soy bean oil, groundnut oil, rapeseed oil, almond oil, palm oil, coconut oil, palm kernel oil and more of the like.

Any desired blends of such oil and wax components are also selected to be employed in the context of the present invention. It may also be preferable, where appropriate, to employ waxes, for example cetyl palmitate, as the sole lipid component of the oily phase.

The oil component is preferably selected from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

Of the hydrocarbons, paraffin oil, squalane and squalene are advantageously to be used in the context of the present invention.

The oil component can furthermore advantageously have a content of cyclic or linear silicone oils or consist entirely of such oils, although it is preferable to use an additional content of other oily phase components in addition to the silicone oil or the silicone oils.

Cyclomethicone (octamethylcyclotetrasiloxane) is preferably employed as the silicone oil to be used according to the invention. However, other silicone oils are also advantageously to be used in the context of the present invention, for example hexamethylcyclotrisiloxane, polydimethylsiloxane and poly(methylphenylsiloxane).

Mixtures of cyclomethicone and isotridecyl isononanoate and of cyclomethicone and 2-ethylhexyl isostearate are furthermore particularly advantageous.

The oil component is furthermore preferably selected from the group consisting of phospholipids. Phospholipids are phosphoric acid esters of acylated glycerols. Among the phosphatidylcholines, for example, the lecithins are of the greatest importance, these being distinguished by the general structure 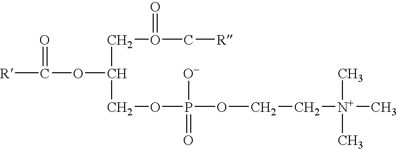
wherein R and R″ typically represent unbranched aliphatic radicals having 15 or 17 carbon atoms and up to 4 cis double bonds.

According to the invention, the oils can be employed by themselves or in a mixture.

The pyrogenically prepared silicas which can be used according to the invention can be prepared by flame hydrolysis of chlorosilanes and are available, for example, under the trade names AEROSIL®, Cab-O-Sil® and Wacker® HDK.

All hydrophilic and hydrophobic pyrogenic silicas, which can optionally be further modified by a chemical or mechanical after-treatment, such as, for example, compaction, granulation or grinding, can be employed. AEROSIL® 150, 200, 255, 300, 380, R 972, R 974, R 805, R 812, R 812 S, R 816, R 8200, Cab-O-Sil® LM-5, M-5, MS-7, H-5, EH-5, TS 610, A 15, TS 620, TS 530, Wacker HDK® V15P, N20, T 30, T 40, H15, H₂O, H30, H18 and H2000 are particularly preferred.

The pyrogenic silicon dioxide is prepared by a procedure in which a volatile silicon compound is injected into an oxyhydrogen gas flame of hydrogen and air. Silicon tetrachloride is used in most cases. This substance hydrolyses to silicon dioxide and hydrochloric acid under the influence of the water formed during the oxyhydrogen gas reaction. After leaving the flame the silicon dioxide enters into a so-called coagulation zone, in which the silicon dioxide primary particles and primary aggregates agglomerate. The product present as a type of aerosol in this stage is separated from the gaseous concomitant substances in cyclones and then after-treated with damp hot air. The residual hydrochloric acid content can be lowered to below 0.025% by this process.

The pyrogenic silicon dioxide can also be silanized. The carbon content of the product is then preferably 0.3 to 15.0 wt. %. Halogenosilanes, alkoxysilanes, silazanes and/or siloxanes can be employed for the silanization.

The following substances can be employed in particular as halogenosilanes:

Halogeno-organosilanes of the type X₃Si(CₙH₂ₙ₊₁)
X=Cl, Br
n=1-20

Halogeno-organosilanes of the type X₂(R′)Si(CₙH₂ₙ₊₁)
X═Cl, Br
R′=alkyl
n=1-20

Halogeno-organosilanes of the type X(R′)₂Si(CₙH₂ₙ₊₁)
X=Cl, Br
R′=alkyl
n=1-20

Halogeno-organosilanes of the type X₃Si(CH₂)ₘ—R′
X=Cl, Br
m=0.1-20
R′=alkyl, aryl (e.g. —C₆H₅)
   —C₄F₉, —OCF₂—CHF—CF₃, —C₆F₁₃, —O—CF₂—CHF₂
   —NH₂, —N₃, —SCN, —CH═CH₂,
   OOC(CH₃)C═CH₂
   —OCH₂—CH(O)CH₂ 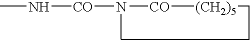
   —NH—COO—CH₃, —NH—COO—CH₂—CH₃, —NH—(CH₂)₃Si(OR)₃
   —Sₓ—(CH₂)₃Si(OR)₃

Halogeno-organosilanes of the type (R)X₂Si(CH₂)ₘ—R′
X=Cl, Br
R=alkyl
m=0.1-20
R′=alkyl, aryl (e.g. —C₆H₅)
—C₄F₉, —OCF₂—CHF—CF₃, —C₆F₁₃, —O—CF₂—CHF₂
—NH₂, —N₃, —SCN, —CH═CH₂,
OOC(CH₃)C═CH₂
—OCH₂—CH(O)CH₂ 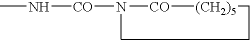
—NH—COO—CH₃, —NH—COO—CH₂—CH₃, —NH—(CH₂)₃Si(OR)₃
—Sₓ—(CH₂)₃Si(OR)₃

Halogeno-organosilanes of the type (R)₂X Si(CH₂)ₘ—R′
X=Cl, Br
R=alkyl
m=0.1-20
R′=alkyl, aryl (e.g. —C₆H₅)
   —C₄F₉, —OCF₂—CHF—CF₃, —C₆F₁₃, —O—CF₂—CHF₂
   —NH₂, —N₃, —SCN, —CH═CH₂,
   OOC(CH₃)C═CH₂
   —OCH₂—CH(O)CH₂ 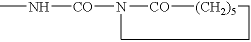
   —NH—COO—CH₃, —NH—COO—CH₂—CH₃, —NH—(CH₂)₃Si(OR)₃
   —Sₓ—(CH₂)₃Si(OR)₃

The following substances can be employed in particular as alkoxysilanes:

Organosilanes of the type (RO)₃Si(CₙH₂ₙ₊₁)
R=alkyl
n=1-20

Organosilanes of the type R′x(RO)_{y}Si(CₙH₂ₙ₊₁)
R=alkyl
R′=alkyl
n=1-20
x+y=3
x=1,2
y=1,2

Organosilanes of the type (RO)₃Si(CH₂)ₘ—R′
R=alkyl
m=0.1-20
R′=alkyl, aryl (e.g. —C₆H₅)
   —C₄F₉, OCF₂—CHF—CF₃, —C₆F₁₃, —O—CF₂—CHF₂
   —NH₂, —N₃, —SCN, —CH═CH₂,
   —OOC(CH₃)C═CH₂
   —OCH₂—CH(O)CH₂ 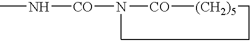
   —NH—COO—CH₃, —NH—COO—CH₂—CH₃, —NH—(CH₂)₃ Si(OR)₃
   —Sₓ—(CH₂)₃Si(OR)₃

Organosilanes of the type (R″)ₓ(RO)_{y}Si(CH₂)ₘ—R′
R″=alkyl
x+y=2
x=1,2
y=1,2
R′=alkyl, aryl (e.g. —C₆H₅)
   —C₄F₉, —OCF₂—CHF—CF₃, —C₆F₁₃, —O—CF₂—CHF₂
   —NH₂, —N₃, —SCN, —CH═CH₂,
   —OOC(CH₃)C═CH₂
   —OCH₂—CH(O)CH₂ 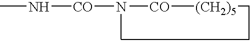
   —NH—COO—CH₃, —NH—COO—CH₂—CH₃, —NH—(CH₂)₃ Si(OR)₃
   —Sₓ—(CH₂)₃Si(OR)₃

The silane Si 108 [(CH₃O)₃—S₁-C₈H₁₇] trimethoxyoctylsilane can preferably be employed as the silanizing agent.

The following substances can be employed in particular as silazanes:

Silazanes of the type: 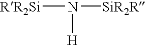
R=alkyl
R′=alkyl, vinyl
   and, for example, hexamethyldisilazane.

The following substances can be employed in particular as siloxanes: Cyclic polysiloxanes of the type D 3, D 4, D 5, e.g. octamethylcyclotetrasiloxane=D 4

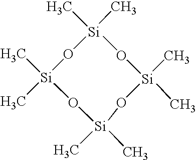

Polysiloxanes or silicone oils of the type: 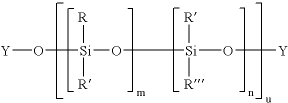
R=alkyl, aryl, (CH₂)ₙ—NH₂, H
R′=alkyl, aryl, (CH₂)ₙ—NH₂, H
R″=alkyl, aryl, (CH₂)ₙ—NH₂, H
R′″=alkyl, aryl, (CH₂)ₙ—NH₂, H
Y═CH₃, H, CₙH₂ₙ₊₁ where n=1-20
Y=Si(CH₃)₃, Si(CH₃)₂H
   Si(CH₃)₂OH, Si(CH₃)₂(OCH₃)
   Si(CH₃)₂(CₙH₂ₙ₊₁) where n=1-20
m=0,1,2,3, . . . ∞
n=0,1,2,3, . . . ∞
u=0,1,2,3, . . . ∞

The silanization can be carried out by a procedure in which the pyrogenic silicon dioxide is sprayed with the silanizing agent, which can optionally be dissolved in an organic solvent, such as, for example, ethanol, and the mixture is then heat-treated at a temperature of 105 to 400° C. over a period of 1 to 6 h.

An alternative method of the silanization can be carried out by a procedure in which the pyrogenic silicon dioxide is treated with the silanizing agent in vapour form and the mixture is then heat-treated at a temperature of 200 to 800° C. over a period of 0.5 to 6 h. The heat treatment can be carried out under an inert gas, such as, for example, nitrogen.

The silanization can be carried out continuously or batchwise in heatable mixers and dryers with spray devices. Suitable devices can be, for example: plough share mixers or plate, fluidized bed or flow-bed dryers.

Pyrogenic metal oxides, such as, for example, titanium dioxide, aluminium oxide and Si/Ti, Si/Al and Si/Al/Ti mixed oxides, can furthermore be employed.

The shower oil gels according to the invention can comprise, in addition to the constituents mentioned, water and further additives usual in cosmetics, such as, for example, emulsifiers, thickeners, solubilizing agents, perfume, dyestuffs, deodorants, antimicrobial substances, re-oiling agents, complexing and sequestering agents, pearlescent agents, plant extracts, vitamins, active compounds and the like. The shower oil gels according to the invention can optionally also comprise one or more emulsifiers. A list of suitable emulsifiers is known, for example, in EP 0 867 176 (page 4, line 21 to page 5, line 46).

The formulations according to the invention optionally also comprise one or more emulsifiers. These are preferably selected from the group consisting of
the fatty alcohol ethoxylates of the general formula R—O—(—CH₂—CH₂—O—)ₙ—H, wherein R represents a branched or unbranched alkyl, aryl or alkenyl radical and n represents a number from 10 to 50,
the ethoxylated wool wax alcohols,
the polyethylene glycol ethers of the general formula R—O—(—CH₂—CH₂—O—)ₙ—R′, wherein R and R′ independently of one another represent branched or unbranched alkyl or alkenyl radicals and n represents a number from 10 to 80,
the fatty acid ethoxylates of the general formula R—COO—(—CH₂—CH₂—O—)ₙ—H, wherein R represents a branched or unbranched alkyl or alkenyl radical and n represents a number from 10 to 40,
the etherified fatty acid ethoxylates of the general formula R—COO—(—CH₂—CH₂—O—)ₙ—R′, wherein R and R′ independently of one another represent branched or unbranched alkyl or alkenyl radicals and n represents a number from 10 to 80,
the esterified fatty acid ethoxylates of the general formula R—COO—(—CH₂—CH₂—O—)ₙ—C(O)—R′, wherein R and R′ independently of one another represent branched or unbranched alkyl or alkenyl radicals and n represents a number from 10 to 80,
the polyethylene glycol glycerol fatty acid esters of saturated and/or unsaturated, branched and/or unbranched fatty acids having a degree of ethoxylation of between 3 and 50,
the ethoxylated sorbitan esters having a degree of ethoxylation of 3 to 100,
the cholesterol ethoxylates having a degree of ethoxylation of between 3 and 50,
the ethoxylated triglycerides having a degree of ethoxylation of between 3 and 150,
the alkyl ether-carboxylic acids of the general formula R—O—(—CH₂—CH₂—O—)ₙ—CH₂—COOH and cosmetically or pharmaceutically acceptable salts thereof, wherein R represents a branched or unbranched alkyl or alkenyl radical having 5-30 C atoms and n represents a number from 5 to 30,
the polyoxyethylene sorbitol fatty acids esters based on branched or unbranched alkanoic or alkenoic acids and having a degree of ethoxylation of 5 to 100, for example of the sorbeth type,
the alkyl ether sulfates, or the acids on which these sulfates are based, of the general formula R—O—(—CH₂—CH₂—O—)ₙ—SO₃—H with cosmetically or pharmaceutically acceptable cations, wherein R represents a branched or unbranched alkyl or alkenyl radical having 5-30 C atoms and n represents a number from 1 to 50.
the fatty alcohol propoxylates of the general formula R—O—(—CH₂—CH(CH₃)—O—)ₙ—H, wherein R represents a branched or unbranched alkyl or alkenyl radical and n represents a number from 10 to 80,
the polypropylene glycol ethers of the general formula R—O—(—CH₂—CH(CH₃)—O—)ₙR′, wherein R and R′ independently of one another represent branched or unbranched alkyl or alkenyl radicals and n represents a number from 10 to 80,
the propoxylated wool wax alcohols,
the etherified fatty acid propoxylates of the general formula R—COO—(—CH₂—CH(CH₃)—O—)ₙ—R′, wherein R and R′ independently of one another represent branched or unbranched alkyl or alkenyl radicals and n represents a number from 10 to 80,
the esterified fatty acid propoxylates of the general formula R—COO—(—CH₂—CH(CH₃)—O—)ₙ—C(O)—R′, wherein R and R′ independently of one another represent branched or unbranched alkyl or alkenyl radicals and n represents a number from 10 to 80,
the fatty acid propoxylates of the general formula R—COO—(—CH₂—CH(CH₃)—O—)ₙ—H, wherein R represents a branched or unbranched alkyl or alkenyl radical and n represents a number from 10 to 80,
the polypropylene glycol glycerol fatty acid esters of saturated and/or unsaturated, branched and/or unbranched fatty acids having a degree of propoxylation of between 3 and 80,
the propoxylated sorbitan esters having a degree of propoxylation of 3 to 100,
the cholesterol propoxylates having a degree of propoxylation of 3 to 100,
the propoxylated triglycerides having a degree of propoxylation of 3 to 100,
the alkyl ether-carboxylic acids of the general formula R—O—(—CH₂—CH(CH₃)O—)ₙ—CH₂—COOH and cosmetically or pharmaceutically acceptable salts thereof, wherein R represents a branched or unbranched alkyl or alkenyl radical and n represents a number from 3 to 50,
the alkyl ether sulfates, or the acids on which these sulfates are based, of the general formula R—O—(—CH₂—CH(CH₃)—O—)ₙ—SO₃—H with cosmetically or pharmaceutically acceptable cations, wherein R represents a branched or unbranched alkyl or alkenyl radical having 5-30 C atoms and n represents a number from 1 to 50,
the fatty alcohol ethoxylates/propoxylates of the general formula R—O—Xₙ—Yₘ—H, wherein R represents a branched or unbranched alkyl or alkenyl radical, and wherein X and Y are not identical and in each case represent either an oxyethylene group or an oxypropylene group and n and m independently of one another represent numbers from 5 to 50,
the polypropylene glycol ethers of the general formula R—O—Xₙ—Yₘ—R′, wherein R and R′ independently of one another represent branched or unbranched alkyl or alkenyl radicals, and wherein X and Y are not identical and in each case represent either an oxyethylene group or an oxypropylene group and n and m independently of one another represent numbers from 5 to 100,
the etherified fatty acid propoxylates of the general formula R—COO—Xₙ—Yₘ—R′, wherein R and R′ independently of one another represent branched or unbranched alkyl or alkenyl radicals, and wherein X and Y are not identical and in each case represent either an oxyethylene group or an oxypropylene group and n and m independently of one another represent numbers from 5 to 100,
the fatty acid ethoxylates/propoxylates of the general formula R—COO—Xₙ—Yₘ—H, wherein R represents a branched or unbranched alkyl or alkenyl radical, and wherein X and Y are not identical and in each case represent either an oxyethylene group or an oxypropylene group and n and m independently of one another represent numbers from 5 to 50.

It is advantageous, in particular, to choose solubilizing agents from the group consisting of polyoxyethylene/polyoxypropylene block copolymers. Such block copolymers are known by the name “poloxamers” and are distinguished by the following structure:

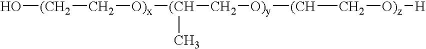

In this formula, x advantageously assumes values between 2 and 20. y advantageously assumes values between 10 and 50. z advantageously assumes values between 2 and 20.

If formulations according to the present invention are to comprise further surfactants in addition to the surfactants according to the invention, it is preferable to choose the concentration thereof at not greater than 5 wt. %, based on the weight of the total composition.

If vitamin A or vitamin A derivatives or carotenes or derivatives thereof are the antioxidant or antioxidants, it is advantageous to choose the particular concentrations thereof from the range of 0.001-10 wt. %, based on the total weight of the formulation.

Antioxidants which are suitable or usual in cosmetic and/or dermatological uses can also be used according to the invention (cf. likewise EP 0 867 176, page 5, line 52 to page 6, line 22).

Amino acids (for example glycine, histidine, tyrosine, tryptophan) and derivatives thereof, imidazoles (for example urocanic acid) and derivatives thereof, peptides, such as D,L-camosine, D-camosine, L-camosine and derivatives thereof (for example anserine), carotenoids, carotenes (for example α-carotene, β-carotene, lycopene) and derivatives thereof, liponic acid and derivatives thereof (for example dihydroliponic acid), aurothioglucose, propylthiouracil and other thiols (for example thioredoxin, glutathione, cysteine, cystine, cystamine and glycosyl, N-acetyl, methyl, ethyl, propyl, amyl, butyl and lauryl, palmitoyl, oleyl, gamma-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example buthionine sulfoximines, homocysteine sulfoximine, buthionine sulfones, penta-, hexa-, heptathionine sulfoximine) in very low tolerated dosages (for example pmol to μmol/kg), furthermore (metal) chelators (for example α-hydroxy-fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example gamma-linolenic acid, linoleic acid, oleic acid), folic acid and derivatives thereof, ubiquinone and ubiquinol and derivatives thereof, vitamin C and derivatives (for example ascorbyl palmitates, Mg ascorbyl phosphates, ascorbyl acetates), tocopherols and derivatives (for example vitamin E acetate), vitamin A and derivatives (vitamin A palmitate) and coniferyl benzoate of benzoin resin, rutic acid and derivatives thereof, ferulic acid and derivatives thereof, butylhydroxytoluene, butylhydroxyanisole, nordihydroguaiac resin acid, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example selenium-methionine), stilbenes and derivatives thereof (for example stilbene oxide, trans-stilbene oxide) and the derivatives of these active compounds mentioned which are suitable according to the invention (salts, esters, ethers, sugars, nucleotides, nucleosides, peptides and lipids).

Oil-soluble antioxidants can be particularly advantageously employed in the context of the present invention. The amount of antioxidants (one or more compounds) in the formulations is preferably 0.001 to 30 wt. %, particularly preferably 0.05-20 wt. %, in particular 1-10 wt. %, based on the total weight of the formulation. If vitamin E and/or derivatives thereof are the antioxidant or antioxidants, it is advantageous to choose the particular concentrations thereof from the range of 0.001-10 wt. %, based on the total weight of the formulation.

It has been found, surprisingly, that the viscosity of shower oil formulations can already be increased considerably by the addition of relatively small concentrations of pyrogenic silica. Products which have a viscosity comparable to that of shower gels and at the same time pseudoplastic flow properties can be prepared in this manner. A further advantage is that the shower oil gels according to the invention are transparent. This is not possible with known gel-forming agents, such as, for example, metal soaps, waxes and/or laminar silicates (e.g. bentones).

Surprisingly, the foaming power of the shower oil gels according to the invention can also be adjusted in a controlled manner by the type and concentration of the pyrogenic silica.

A further advantage is the good feeling of the products according to the invention on the skin and the high re-oiling power. This was surprising since pyrogenically prepared silicas in cosmetics often cause a dry and dull feeling on the skin.

### EXAMPLES

| | | Without | | | | | |
|---|---|---|---|---|---|---|---|
| Raw materials | INCI name | AE | 200-20A | 200-25A | 200-30A | 972-20A | 972-23A |
| Soy bean oil | Glycine Soja | 28.30 | 26.30 | 25.80 | 25.30 | 26.30 | 26.00 |
| Zetesol 100 | MIPA-Laureth-Sulfate | 57.00 | 57.00 | 57.00 | 57.00 | 57.00 | 57.00 |
| | & Laureth-4 & | | | | | | |
| | Cocamide DEA | | | | | | |
| Castor oil | Ricinus Communis | 13.00 | 13.00 | 13.00 | 13.00 | 13.00 | 13.00 |
| | (Castor) Seed Oil | | | | | | |
| Nature Therapy | Perfume (Fragrance) | 1.00 | 1.00 | 1.00 | 1.00 | 1.00 | 1.00 |
| D1130A | | | | | | | |
| Oxynex K liquid * | Ascorbyl Palmitate & | 0.20 | 0.20 | 0.20 | 0.20 | 0.20 | 0.20 |
| | Tocopherol & Ascorbic | | | | | | |
| | Acid & Citric Acid & | | | | | | |
| | PEG-8 | | | | | | |
| alpha-Tocopherol E | Tocopherol | 0.50 | 0.50 | 0.50 | 0.50 | 0.50 | 0.50 |
| 307 | | | | | | | |
| Aerosil 200 | Silica | — | 2.00 | 2.50 | 3.00 | — | — |
| Aerosil R 972 | Silica Dimethyl Silylate | — | — | — | — | 2.00 | 2.30 |
| Total | | 100.00 | 100.00 | 100.00 | 100.00 | 100.00 | 100.00 |

The amounts of oils, Zetesol 100, perfume oil and Oxynex K liquid necessary for a 500 g batch are initially introduced into a 1,000 ml glass beaker. The corresponding amount of AEROSIL 200 or AEROSIL R 972 was stirred in with a Pendraulik dissolver, Stephan, Hameln (Germany), disc diameter 6 cm, at 470 rpm. This mixture is then subjected to dispersion for 10 minutes at 3,000 rpm.

After storage for 24 hours, the viscosity of the gels is determined with a Brookfield RVDV-III+cP rheometer with a small sample adapter, spindle SC4-27 at 20 rpm.

To determine the foaming power of a shower gel, 0.1 g is added to 30 ml of deionized water in a 100 ml measuring cylinder. The measuring cylinder is closed with a stopper and manually shaken horizontally for one minute. The standing cylinder is then left to stand for two minutes, before the foam height is read off.

Two commercial products (Nivea shower oil, Beiersdorf; Ombia Med, Aldi) are also included in the investigation as comparison products.

| | Without | | | | | | | Ombia |
|---|---|---|---|---|---|---|---|---|
| Example | AE | 200-20A | 200-25A | 200-30A | 972-20A | 972-23A | Nivea | Med |
| Viscosity (mPas) | 175 | 388 | 588 | 825 | 271 | 325 | 125 | 119 |
| Foam height | 110 | 100 | 93 | 88 | 41 | 41 | 100 | 105 |
| (cm) | | | | | | | | |

The addition of pyrogenic silica leads to a significant increase in the viscosity. The viscosity and foaming power can be controlled in this context via the type and concentration of the pyrogenic silica. While hydrophilic AEROSIL® 200 has virtually no influence on the foam formation, hydrophobic AEROSIL® R 972 has a defoaming effect.

Further variations and modifications of the foregoing will be apparent to those skilled in the art and are intended to be encompassed by the claims appended hereto.

German prior application No. 10 2004 009 812.3 of Feb. 28, 2004, is relied on and incorporated herein by reference.

1. A shower oil gel composition comprising up to 70 wt. % of one or more oil-soluble surfactants, 5 to 70 wt. % of one or more oil components, 0.1 to 25 wt. % of one or more pyrogenic silicas and optionally at least one of a cosmetic, and pharmaceutical auxiliary substance, additive and active compound.

2. A shower oil gel composition according to claim 1 having a content of one or more oil-soluble surfactants of more than 20 wt. %.

3. The shower oil gel composition according to claim 1, wherein the one or more surfactants is present in an amount of 20 to 70 wt. % of the composition.

4. The shower oil gel composition according to claim 1, which is transparent.

5. A shower oil gel composition comprising 5 to 70 wt. % of at least one surfactant selected from the group consisting of wash-active anionic, cationic, amphoteric and nonionic surfactants, 5 to 70 wt. % of at least one oil compound selected from the group consisting of polar oils, paraffin oil, silicone oils and synthetic oils, and 0.1 to 25 wt. % of at least one pyrogenic silica.

6. The shower oil gel composition according to claim 5, wherein the surfactant is a member selected from the group consisting of fatty alcohol sulphates and amides thereof, fatty alcohol ether sulfates and amides thereof, fatty alcohol ethoxylates, and fatty acid alkanol amides.

7. The shower oil gel composition according to claim 5, which is transparent.

8. A cosmetic or dermatological cleansing formulation comprising up to 70 wt. % of at least one oil-soluble surfactant,
5 to 70 wt. % of at least one oil component selected from the group consisting of esters of saturated or unsaturated alkanecarboxylic acids containing 3 to 30 carbon atoms, and saturated or unsaturated alcohols containing 3 to 30 carbon atoms, esters of aromatic carboxylic acids containing 3 to 30 carbon atoms and saturated or unsaturated alcohols containing 3 to 30 carbon atoms, hydrocarbons, hydrocarbon waxes, silicon oils, dialkylethers, triglycerides, and phospholipids,
0.1 to 25 wt. % of at least one pyrogenic silica, optionally modified by chemical or mechanical treatment
and optionally an emulsifier, thickener, solubilizing agent, perfume, dyestuff, deodorant, antimicrobial agent, re-oiling agent, complexing and sequestering agent, pearlescent agent, plant extract or vitamin.

9. The cosmetic or dermatological cleansing formulation of claim 8, which is transparent.
